(19) United States
(12) Patent Application Publication
(10) Pub. No.: US 2025/0057538 A1
(43) Pub. Date: Feb. 20, 2025

(54) DELIVERY DEVICE FOR OCCLUSIVE IMPLANTS

(71) Applicant: Boston Scientific Scimed, Inc., Maple Grove, MN (US)

(72) Inventors: Sara Reda Eskander, Plymouth, MN (US); David John Onushko, Maple Grove, MN (US); Daniel James Viola, Minneapolis, MN (US); Andrea Goering, Blaine, MN (US)

(73) Assignee: Boston Scientific Scimed, Inc., Maple Grove, MN (US)

(21) Appl. No.: 18/805,800

(22) Filed: Aug. 15, 2024

Related U.S. Application Data
(60) Provisional application No. 63/533,027, filed on Aug. 16, 2023.

(51) Int. Cl. A61B 17/12; A61B 17/00

(52) U.S. Cl. CPC A61B 17/12122; A61B 17/00234; A61B 17/12031; A61B 17/12172; A61B 2017/00243; A61B 2017/00305; A61B 2017/00477; A61B 2017/12054

(57) ABSTRACT

Delivery devices for an occlusive implant and method for making and using delivery devices are disclosed. An example delivery device may include a delivery sheath having a proximal region and a distal tip region. A reinforcing member may extend along the proximal region. The distal tip region may include a grooved distal section. The grooved distal section may be configured to shift between a delivery configuration and an expanded configuration. A core member may be slidably disposed within the delivery sheath. An occlusive implant may be releasably coupled to the core member.

### CROSS-REFERENCE TO RELATED APPLICATIONS

This application claims the benefit of U.S. Provisional Patent Application Ser. No. 63/533,027, filed Aug. 16, 2023, which is incorporated herein by reference.

### TECHNICAL FIELD

The present disclosure pertains to medical devices, and methods for manufacturing medical devices. More particularly, the present disclosure pertains to delivery devices for occlusive implants.

### BACKGROUND

A wide variety of medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. A delivery device for an occlusive implant is disclosed. The delivery device comprises: a delivery sheath having a proximal region and a distal tip region; a reinforcing member extending along the proximal region; wherein the distal tip region has a grooved distal section, the grooved distal section being configured to shift between a delivery configuration and an expanded configuration; a core member slidably disposed within the delivery sheath; and an occlusive implant releasably coupled to the core member.

Alternatively or additionally to any of the embodiments above, the grooved distal section is fluted.

Alternatively or additionally to any of the embodiments above, the grooved distal section includes one or more pleats.

Alternatively or additionally to any of the embodiments above, the grooved distal section includes one or more folds.

Alternatively or additionally to any of the embodiments above, the grooved distal section includes one or more folds when in the delivery configuration.

Alternatively or additionally to any of the embodiments above, the grooved distal section includes one or more axial slits.

Alternatively or additionally to any of the embodiments above, the proximal region includes a proximal outer diameter and wherein the grooved distal section has distal outer diameter that is substantially the same as or smaller than the proximal outer diameter when in the delivery configuration.

Alternatively or additionally to any of the embodiments above, the proximal region includes a proximal outer diameter and wherein the grooved distal section has distal outer diameter that is substantially the same as the proximal outer diameter when in the delivery configuration.

Alternatively or additionally to any of the embodiments above, the proximal region includes a proximal outer diameter and wherein the grooved distal section has distal outer diameter that is larger than the proximal outer diameter when in the delivery configuration.

Alternatively or additionally to any of the embodiments above, the proximal region includes a proximal outer diameter and wherein the grooved distal section has distal outer diameter that is smaller than the proximal outer diameter when in the delivery configuration.

Alternatively or additionally to any of the embodiments above, the proximal region includes a proximal outer diameter and wherein the grooved distal section has distal outer diameter that is larger than the proximal outer diameter when in the expanded configuration.

A delivery device for an occlusive implant is disclosed. The delivery device comprises: a delivery sheath having a proximal region and a distal tip region; wherein the distal tip region includes a grooved distal section; wherein the grooved distal section is configured to allow the distal tip region to shift between a delivery configuration and an expanded configuration; and a proximal reinforcing member disposed along the proximal region.

Alternatively or additionally to any of the embodiments above, the grooved distal section is fluted.

Alternatively or additionally to any of the embodiments above, the grooved distal section includes one or more pleats.

Alternatively or additionally to any of the embodiments above, the grooved distal section includes one or more folds.

Alternatively or additionally to any of the embodiments above, the grooved distal section includes one or more folds when in the delivery configuration.

Alternatively or additionally to any of the embodiments above, the grooved distal section includes one or more axial slits.

Alternatively or additionally to any of the embodiments above, the proximal region includes a proximal outer diameter and wherein the grooved distal section has distal outer diameter that is substantially the same as or smaller than the proximal outer diameter when in the delivery configuration.

Alternatively or additionally to any of the embodiments above, the proximal region includes a proximal outer diameter and wherein the grooved distal section has distal outer diameter that is substantially the same as the proximal outer diameter when in the delivery configuration.

Alternatively or additionally to any of the embodiments above, the proximal region includes a proximal outer diameter and wherein the grooved distal section has distal outer diameter that is larger than the proximal outer diameter when in the delivery configuration.

Alternatively or additionally to any of the embodiments above, the proximal region includes a proximal outer diameter and wherein the grooved distal section has distal outer diameter that is smaller than the proximal outer diameter when in the delivery configuration.

Alternatively or additionally to any of the embodiments above, the proximal region includes a proximal outer diameter and wherein the grooved distal section has distal outer diameter that is larger than the proximal outer diameter when in the expanded configuration.

Alternatively or additionally to any of the embodiments above, further comprising a core member slidably disposed within the delivery sheath and an occlusive implant releasably coupled to the core member.

A method for implanting an occlusive implant into a left atrial appendage is disclosed. The method comprises: advancing a delivery device to a position adjacent to the left atrial appendage, the delivery device comprising: a delivery sheath having a proximal region and a distal tip region, a reinforcing member extending along the proximal region, wherein the distal tip region includes a grooved distal section, the grooved distal section being configured to shift between a delivery configuration and an expanded configuration, a core member slidably disposed within the delivery sheath, and an occlusive implant releasably coupled to the core member; and advancing the core member such that the occlusive implant advances out from the grooved distal section.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a side view of an example delivery system.

FIG. 2 is a side view of an example delivery system.

FIG. 3 is a side view of a portion of an example delivery system.

FIG. 4 is an orthogonal view of a portion of an example delivery system.

FIG. 5 is a schematic side view of a portion of an example delivery sheath.

FIG. 6 is a schematic side view of a portion of an example delivery sheath.

FIG. 7 is a schematic side view of a portion of an example delivery sheath.

FIG. 8 is a schematic side view of a portion of an example delivery sheath.

FIG. 9 is a schematic side view of a portion of an example delivery sheath.

FIG. 10 is a schematic side view of a portion of an example delivery sheath.

FIG. 11 is a schematic side view of a portion of an example delivery sheath.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

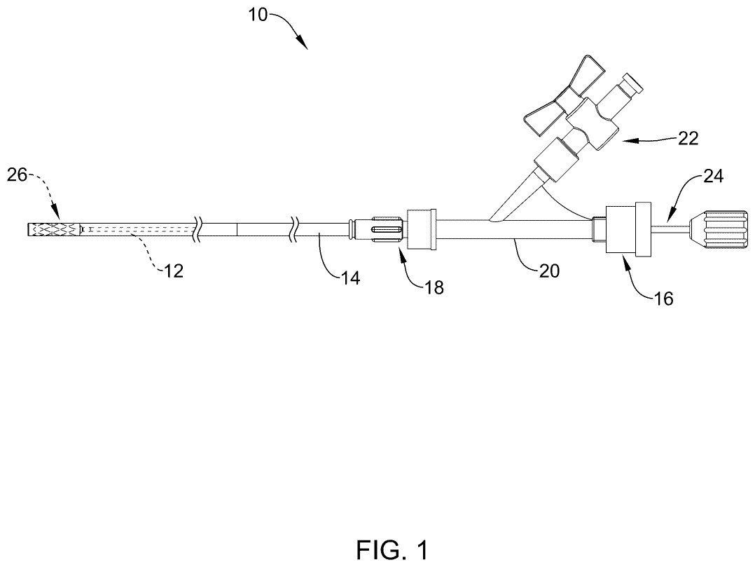

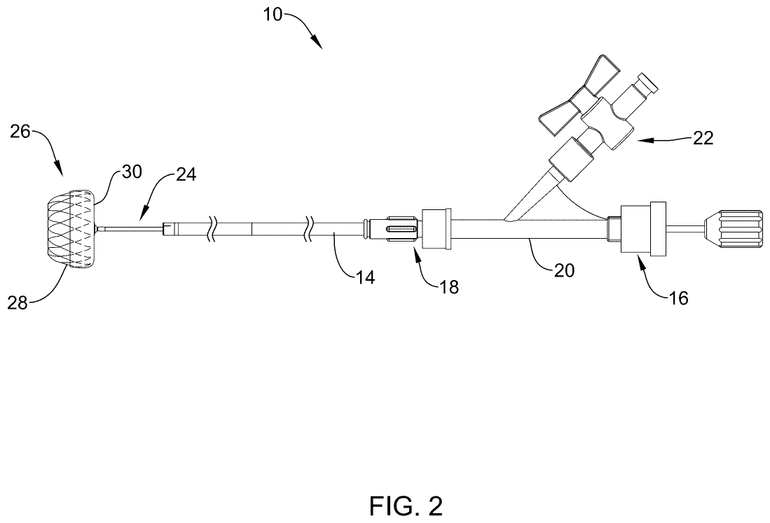

FIGS. 1-2 schematically illustrate selected components and/or arrangements of an occlusive implant system. It should be noted that in any given figure, some features of the occlusive implant system may not be shown, or may be shown schematically, for simplicity. Additional details regarding some of the components of the occlusive implant system may be illustrated in other figures in greater detail. The occlusive implant system may be used to deliver and/or deploy a variety of medical implants (e.g., a cardiovascular implant, an occlusive implant, etc.) to one or more locations within the anatomy, including but not limited to, in some embodiments, the heart and/or the left atrial appendage. In the interest of clarity, the following discussion refers to an occlusive implant, but other medical implants may be used and/or considered with the occlusive implant system.

The occlusive implant system may include a delivery system 10 including a delivery sheath 14 having a delivery lumen 12 extending proximally from a distal end of the delivery sheath 14. In one example, the delivery lumen 12 extends from a proximal opening to a distal opening of the delivery sheath 14. The delivery system 10 may include a proximal hub 16. In some embodiments, the delivery system may include a mid-hub 18. In some embodiments, the delivery system 10 may include a mid-shaft 20 extending from the proximal hub 16 to the mid-hub 18. In some embodiments, the delivery sheath 14 may extend distally from the mid-hub 18. Other configurations are also contemplated. In some embodiments, the delivery system 10 may include a side port 22. In some embodiments, the side port 22 may be in communication with the mid-shaft 20. Other configurations are also contemplated. In some embodiments, the delivery system 10 and/or the delivery lumen 12 may include a proximal segment (not shown) extending within and/or through the mid-hub 18, the mid-shaft 20, and the proximal hub 16. In some embodiments, the proximal segment may be in fluid communication with and/or may be an extension of the delivery lumen 12 of the delivery sheath 14. In some embodiments, the side port 22 may be in fluid communication with the proximal segment and/or the delivery lumen 12.

The occlusive implant system and/or the delivery system 10 may include a core member or core wire 24 slidably and/or rotatably disposed within the delivery lumen 12 (and the proximal segment, where present). The occlusive implant system may include an occlusive implant 26, which may be configured for implantation within a left atrial appendage, releasably engaged with and/or releasably attached to a distal end of the core wire 24. In at least some embodiments, the occlusive implant 26 may be a left atrial appendage closure device. In some embodiments, a proximal end of the core wire 24 may extend proximally of a proximal end of the delivery sheath 14 and/or the proximal opening of the delivery lumen 12 for manual manipulation by a clinician or practitioner. In at least some embodiments, the delivery sheath 14 may comprise and/or may be formed from a polymeric material. In some embodiments, the delivery sheath 14 may comprise and/or may be formed from a plurality of polymeric materials. In some embodiments, the delivery sheath may comprise and/or may be formed from a combination of metallic and polymeric materials. In some embodiments, the delivery sheath 14 may include a reinforcing element, such as a mesh, a coil, a braid, etc., formed therein, embedded therein, attached thereto, etc. along at least a portion of a length of the delivery sheath 14. Other configurations are also contemplated. Some suitable, but non-limiting, examples of materials for the occlusive implant system, the core wire 24, and/or the delivery sheath 14, etc., including but not limited to metallic materials, polymeric materials, etc., are discussed below.

The occlusive implant 26 may include an expandable framework 28 (e.g., FIG. 2) configured to shift between a delivery configuration (e.g., FIG. 1), such as when the occlusive implant 26 is disposed within the delivery lumen 12 proximate the distal opening and/or within a distal portion of the delivery lumen 12, and a deployed configuration (e.g., FIG. 2) when the occlusive implant 26 is unconstrained by the delivery sheath 14.

In some embodiments, the expandable framework 28 may comprise a plurality of interconnected struts. In some embodiments, the expandable framework 28 may be compliant or semi-compliant and may generally conform to and/or be configured to sealingly engage with the shape and/or geometry of the left atrial appendage in the deployed configuration.

In some embodiments, a proximal end of the expandable framework 28 may be configured to releasably attach, join, couple, engage, or otherwise connect to the distal end of the core wire 24 (e.g., FIG. 2). In some embodiments, the proximal end of the expandable framework 28 may include a proximal hub coupled and/or non-releasably attached thereto. In some embodiments, the proximal hub may be configured to and/or adapted to releasably couple with, join to, mate with, or otherwise engage a distal end of the core wire 24. Other means of releasably coupling and/or engaging the expandable framework 28 to the distal end of the core wire 24 are also contemplated.

In some embodiments, the occlusive implant 26 may include an occlusive element 30 (e.g., a membrane, a fabric, or a tissue element, etc.) connected to, disposed on, disposed over, disposed about, or covering at least a portion the expandable framework 28. In some embodiments, the occlusive element 30 may be connected to, disposed on, disposed over, disposed about, or cover at least a portion of an outer (or outwardly facing) surface of the expandable framework 28.

In some embodiments, the occlusive element 30 may be permeable or impermeable to blood and/or other fluids, such as water. In some embodiments, the occlusive element 30 may include a polymeric membrane, a metallic or polymeric mesh, a porous or semi-porous filter-like material, or other suitable construction. In some embodiments, the occlusive element 30 prevents thrombi (e.g., blood clots, etc.) from passing through the occlusive element 30 and out of the left atrial appendage into the blood stream. In some embodiments, the occlusive element 30 promotes endothelization after implantation, thereby effectively removing the target site (e.g., the left atrial appendage, etc.) from the patient's circulatory system. Some suitable, but non-limiting, examples of materials for the occlusive element 30 are discussed below.

In some embodiments, the expandable framework 28 and/or the plurality of interconnected struts may be integrally formed and/or cut from a unitary member. In some embodiments, the expandable framework 28 and/or the plurality of interconnected struts may be integrally formed and/or cut from a unitary tubular member and subsequently formed and/or heat set to a desired shape in the deployed configuration. In some embodiments, the expandable framework 28 and/or the plurality of interconnected struts may be integrally formed and/or cut from a unitary flat member or sheet, and then rolled or formed into a tubular structure and subsequently formed and/or heat set to the desired shape in the deployed configuration. Some exemplary means and/or methods of making and/or forming the expandable framework 28 include laser cutting, machining, punching, stamping, electro discharge machining (EDM), chemical dissolution, etc. Other means and/or methods are also contemplated.

In use, the delivery sheath 14 may be advanced and/or navigated to the left atrial appendage to deliver the occlusive implant 26 thereto. In one example, the delivery sheath 14 may be advanced and/or navigated to the left atrial appendage using and/or over a guidewire. For example, the delivery sheath 14 may be advanced to the patient's left atrium and the distal end disposed adjacent to the left atrial appendage with the occlusive implant 26 disposed therein in the delivery configuration. In some embodiments, the delivery sheath 14 may include steering capability. After the distal end of the delivery sheath 14 is disposed adjacent to and/or at the left atrial appendage, the core wire 24 may be advanced distally relative to the delivery sheath 14 to advance the occlusive implant 26 out of the delivery sheath 14, where the occlusive implant 26 may shift to the deployed configuration.

While not expressly illustrated, in some embodiments, the occlusive implant system may further comprise an access device. In some embodiments, the access device may be a bi-directional steerable catheter and/or an intravascular catheter. Examples of intravascular catheters may include, but are not limited to, balloon catheters, atherectomy catheters, device delivery catheters, drug delivery catheters, diagnostic catheters, and guide catheters.

In some embodiments, the access device may be advanced and/or navigated to the left atrial appendage. In one example, the access device may be advanced and/or navigated to the left atrial appendage using and/or over a guidewire. For example, the access device may be advanced to the patient's left atrium and a distal tip disposed adjacent to the left atrial appendage. In some embodiments, the access device may include steering capability. In some embodiments, the delivery system 10 may be inserted through the access device. In some embodiments, the length of the delivery sheath 14 may be substantially equal to the length of the access device. In some embodiments, the length of the delivery sheath 14 may be slightly longer than the access device. During use, the delivery sheath 14 may be advanced within the access device with the occlusive implant 26 disposed therein in the delivery configuration. After the distal end of the delivery sheath 14 is disposed adjacent to and/or at the distal end of the access device, the core wire 24 may be advanced distally relative to the delivery sheath 14 and/or the access device to advance the occlusive implant 26 out of the delivery sheath 14 and the access device, where the occlusive implant 26 may shift to the deployed configuration.

In some embodiments, the delivery system, the delivery sheath 14, and/or the access device may be sized in accordance with its intended use. For example, the delivery system, the delivery sheath 14, and/or the access device can have a length that is in the range of about 10 to about 150 centimeters, about 25 to about 125 centimeters, about 50 to about 100 centimeters, about 25 centimeters to about 50 centimeters, about 50 to about 75 centimeters, about 75 to about 100 centimeters, etc. Other lengths are also contemplated, including but not limited to subsets of ranges disclosed herein. It is further contemplated that the outer diameter of the delivery system, the delivery sheath 14, and/or the access device may vary based on the use or application. In some examples, the outer diameter of the delivery system, the delivery sheath 14, and/or the access device may be about 2 millimeters (mm), about 3 mm (or 9 French), about 3.5 mm, about 4 mm (or 12 French), about 4.5 mm, about 5 mm (or 15 French), about 5.33 mm, about 5.5 mm, about 5.66 mm (or 17 French), about 6 mm, about 6.5 mm, about 7 mm (or 21 French), about 8 mm, or other suitable sizes. In some embodiments, the outer diameter of the delivery system, the delivery sheath 14, and/or the access device may be a maximum of 5.66 mm (17 French) and is preferably smaller than 5.66 mm (17 French). Other configurations are also contemplated. In some embodiments, it is desirable for the outer diameter of the delivery system, the delivery sheath 14, and/or the access device to be as small as possible.

In some instances, during a procedure, it may be necessary to recapture and/or reposition the occlusive implant 26. For example, the initial placement of the occlusive implant 26 may be incorrect and/or inadequate. Accordingly, the delivery sheath 14 and/or the access device may be configured to permit recapture and/or reposition of the occlusive implant 26. Disclosed herein are delivery systems that include a delivery sheath, for example, with proximal region, a garage region, and a distal tip region. These and/or other structures may help to make the delivery sheath more amenable to recapture and/or repositioning of the occlusive implant. Some additional details regarding these and other structures are disclosed herein.

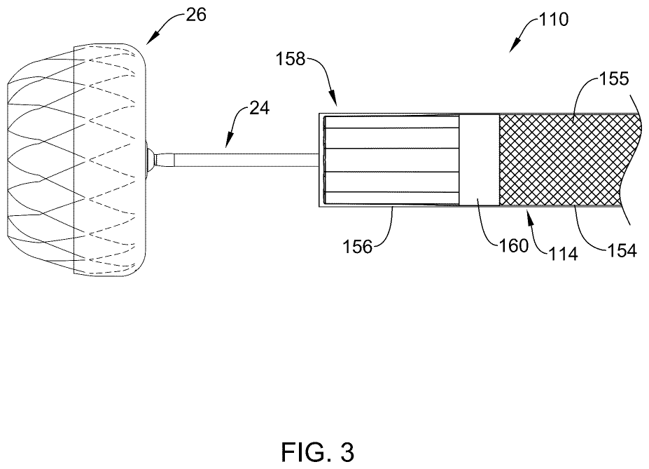

FIGS. 3-5 illustrate a portion of another example delivery device 110 (e.g., for an occlusive implant) that may be similar in form and function to other delivery devices disclosed herein. The delivery device 110 may include a delivery sheath 114 having a proximal region 154 and a distal tip region 156. The proximal region 154 may include a reinforcing member 155. In some instances, the reinforcing member 155 may take the form of a braid. Other reinforcing members are contemplated including coils, meshes, wires, and/or the like. The distal tip region 156 may include a base 160. In some instances, the base 160 may include or otherwise define a radiopaque marker or band that may help to visualize the distal tip region 156 during an intervention.

The distal tip region 156 may include a grooved distal section 158. In general, the grooved distal section 158 (and/or other grooved distal sections disclosed herein) are configured to allow the distal tip region 156 (and/or the grooved distal section 158) to shift between a first or delivery configuration and a second or expanded configuration. The expanded configuration may help an occlusive implant (e.g., such as the occlusive implant 26) to be efficiently guided into the distal tip region 156, for example during a recapture and/or repositioning process. For the purposes of this disclosure, a “grooved” section may be understood to be a section where one or more grooves (e.g., axial and/or circumferential grooves) are formed therein, a section where a portion of the structure is removed or ablated to form a groove or channel therein, a section that is fluted or includes one or more flutes, a section that is pleated (e.g., including pleats where one or more portions of the structure are folded and pleats where one or more portions of the structure are gathered or bunched together), a section that includes one or more folds, a section that includes one or more gathered together portions, a section that includes one or more bunched together portions, combinations thereof, and/or the like. The grooved distal section 158 may be formed using a suitable process. For example, the grooved distal section 158 may be formed by ablating the distal tip region 156, thermally treating the distal tip region 156, stretching the distal tip region 156, molding the distal tip region 156 into the desired shape/configuration, combinations thereof, and/or the like.

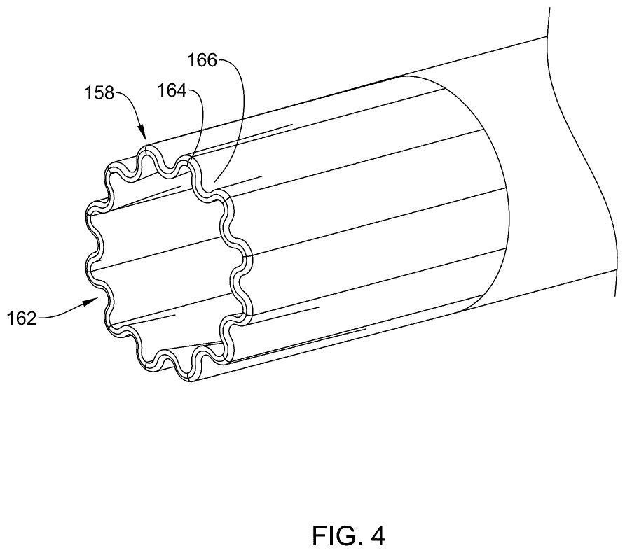

As shown in FIG. 4, the grooved distal section 158 may include or define an undulating end surface 162 that, for example, may define a plurality of peaks or flower-like petals 164. Grooves 166 may be disposed between adjacent peaks 164. In some instances, the structure of the grooved distal section 158 may be described as being fluted or pleated.

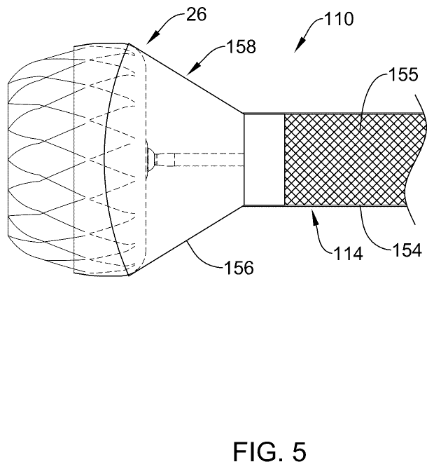

In general, the grooved distal section 158 may be configured to shift between a first or delivery configuration (e.g., as shown in FIGS. 3-4) and a second or expanded configuration (e.g., as shown in FIG. 5). When in the expanded configuration, the grooved distal section 158 may help to hold or maintain the distal tip region 156 into a shape that resembles a funnel and/or otherwise provide a funnel-like shape to the distal tip region 156 (e.g., as shown in FIG. 5). This may help to guide the occlusive implant 26 into the distal tip region 156 (e.g., by actuating the core wire 24), for example when it is desirable to recapture and/or reposition the implant. The funnel-like shape may help to reduce damage to the delivery sheath 114 during a recapture and/or repositioning process. In some instances, when the grooved distal section 158 is in the expanded configuration, the grooves 166 may be smoothed (e.g., the grooved distal section 158 is expanded so that the grooves 166 are merged with the peaks 164 in a manner that smooths out or otherwise removes the grooves 166). When shifting back to the first configuration, the grooved distal section 158 may be configured so that the grooves 166 (and the peaks 164) are re-formed or remembered. Shifting back and/or “remembering” the first configuration may be aided by the grooved distal section 158 including a shape memory material (e.g., a shape memory polymer and/or metal), by the resilience and/or elastic properties of the grooved distal section 158, combinations thereof, and/or the like.

The grooved distal section 158 may have an outer diameter that is substantially the same as the outer diameter of the proximal region 154. For example, the peaks 164 may define the outer diameter of the grooved distal section 158 and the outer diameter may be substantially the same as the outer diameter of the proximal region 154. For example, the outer diameter of the grooved distal section 158 may be substantially the same as the outer diameter of the proximal region 154 when the grooved distal section 158 is in the delivery configuration. Alternatively, the outer diameter of the grooved distal section 158 may be smaller than the outer diameter of the proximal region 154 when the grooved distal section 158 is in the delivery configuration.

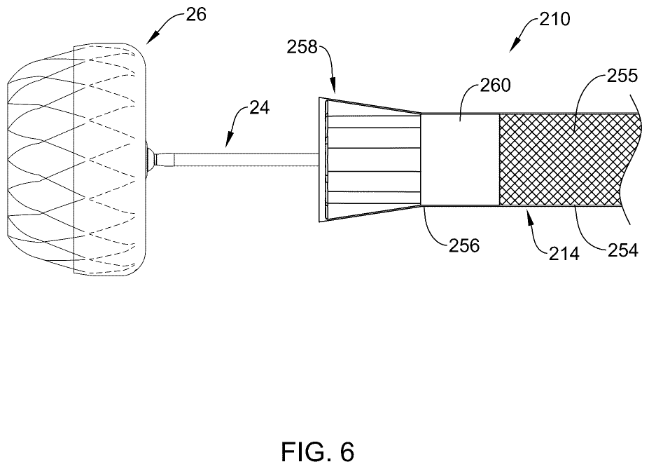

FIGS. 6-8 illustrate a portion of another example delivery device 210 (e.g., for an occlusive implant) that may be similar in form and function to other delivery devices disclosed herein. The delivery device 210 may include a delivery sheath 214 having a proximal region 254 and a distal tip region 256. The proximal region 254 may include a reinforcing member 255. In some instances, the reinforcing member 255 may take the form of a braid. Other reinforcing members are contemplated including coils, meshes, wires, and/or the like. The distal tip region 256 may include a base 260. In some instances, the base 260 may include or otherwise define a radiopaque marker or band that may help to visualize the distal tip region 256 during an intervention.

The distal tip region 256 may include a grooved distal section 258. In general, the grooved distal section 258 (and/or other grooved distal sections disclosed herein) are configured to allow the distal tip region 256 (and/or the grooved distal section 258) to shift between a first or delivery configuration and a second or expanded configuration. The expanded configuration may help an occlusive implant (e.g., such as the occlusive implant 26) to be efficiently guided into the distal tip region 256, for example during a recapture and/or repositioning process.

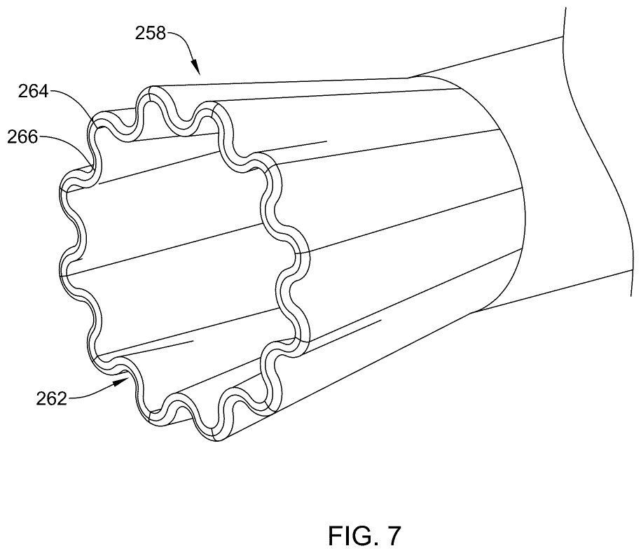

As shown in FIG. 7, the grooved distal section 258 may include or define an undulating end surface 262 that, for example, may define a plurality of peaks or flower-like petals 264. Grooves 266 may be disposed between adjacent peaks 264. In some instances, the structure of the grooved distal section 258 may be described as being fluted or pleated. The grooved distal section 258 may be formed using a suitable process. For example, the grooved distal section 258 may be formed by ablating the distal tip region 256, thermally treating the distal tip region 256, stretching the distal tip region 256, molding the distal tip region 256 into the desired shape/configuration, combinations thereof, and/or the like.

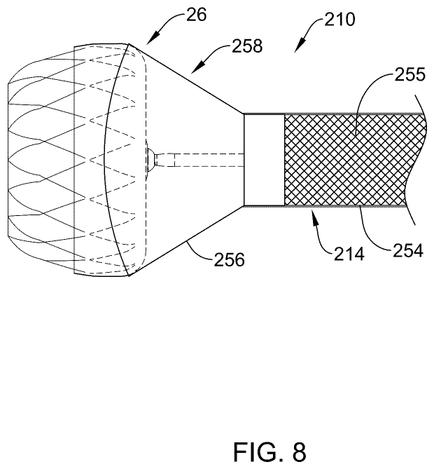

In general, the grooved distal section 258 may be configured to shift between a first or delivery configuration (e.g., as shown in FIGS. 6-7) and a second or expanded configuration (e.g., as shown in FIG. 8). When in the expanded configuration, the grooved distal section 258 may help to hold or maintain the distal tip region 256 into a shape that resembles a funnel and/or otherwise provide a funnel-like shape to the distal tip region 256 (e.g., as shown in FIG. 8). This may help to guide the occlusive implant 26 into the distal tip region 256 (e.g., by actuating the core wire 24), for example when it is desirable to recapture and/or reposition the implant. The funnel-like shape may help to reduce damage to the delivery sheath 214 during a recapture and/or repositioning process. In some instances, when the grooved distal section 258 is in the expanded configuration, the grooves 266 may be smoothed (e.g., the grooved distal section 258 is expanded so that the grooves 266 are merged with the peaks 264 in a manner that smooths out or otherwise removes the grooves 266). When shifting back to the first configuration, the grooved distal section 258 may be configured so that the grooves 266 (and the peaks 264) are re-formed or remembered. Shifting back and/or “remembering” the first configuration may be aided by the grooved distal section 258 including a shape memory material (e.g., a shape memory polymer and/or metal), by the resilience and/or elastic properties of the grooved distal section 258, combinations thereof, and/or the like.

The grooved distal section 258 may have an outer diameter that is larger than the outer diameter of the proximal region 254. For example, the peaks 264 may define the outer diameter of the grooved distal section 258 and the outer diameter may be larger than the outer diameter of the proximal region 254. In at least some instances, the outer diameter of the grooved distal section 258 may be larger than the outer diameter of the proximal region 254 when the grooved distal section 258 is in the delivery configuration.

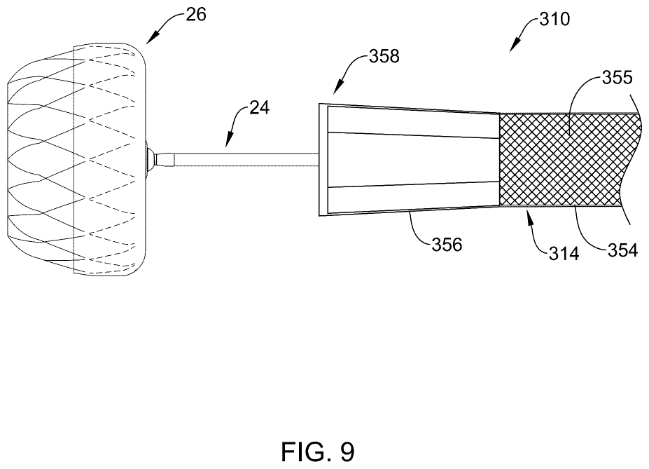

FIGS. 9-11 illustrate a portion of another example delivery device 310 (e.g., for an occlusive implant) that may be similar in form and function to other delivery devices disclosed herein. The delivery device 310 may include a delivery sheath 314 having a proximal region 354 and a distal tip region 356. The proximal region 354 may include a reinforcing member 355. In some instances, the reinforcing member 355 may take the form of a braid. Other reinforcing members are contemplated including coils, meshes, wires, and/or the like. The distal tip region 356 may include a base 260. In some instances, the base 360 may include or otherwise define a radiopaque marker or band that may help to visualize the distal tip region 356 during an intervention.

The distal tip region 356 may include a grooved distal section 358. In general, the grooved distal section 358 (and/or other grooved distal sections disclosed herein) are configured to allow the distal tip region 356 (and/or the grooved distal section 358) to shift between a first or delivery configuration and a second or expanded configuration. The expanded configuration may help an occlusive implant (e.g., such as the occlusive implant 26) to be efficiently guided into the distal tip region 356, for example during a recapture and/or repositioning process.

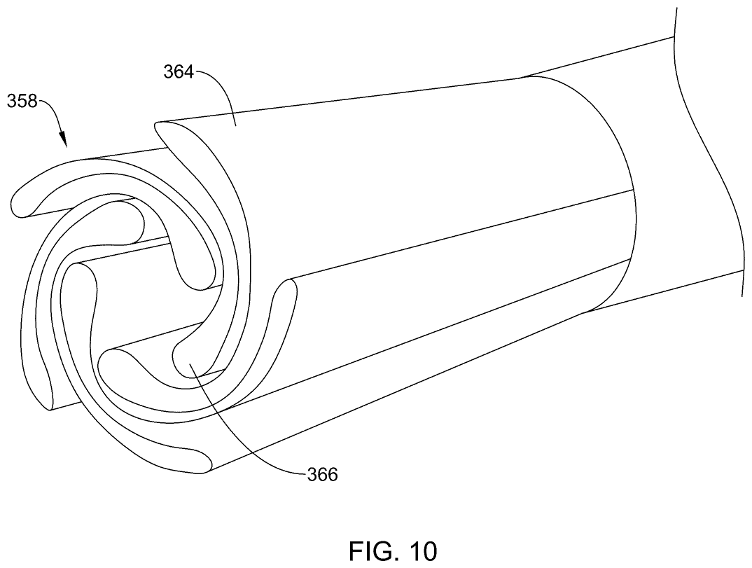

As shown in FIG. 10 the grooved distal section 358 may include or be defined by one or more folds 364 in the grooved distal section 358. For example, the grooved distal section 358 may have a structure that resembles a typical angioplasty balloon (e.g., including a balloon cone or conical section when expanded). The balloon-like structure may be configured to fold into a collapsed structure. Accordingly, the grooves 366 in the grooved distal section 358 may be defined at the base of the folds and/or underneath the folds 364.

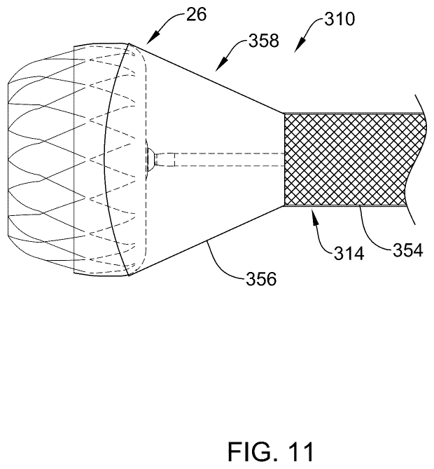

In general, the grooved distal section 358 may be configured to shift between a first or delivery configuration (e.g., as shown in FIGS. 9-10) and a second or expanded configuration (e.g., as shown in FIG. 11). When in the expanded configuration, the grooved distal section 358 may help to hold or maintain the distal tip region 356 into a shape that resembles a funnel and/or otherwise provide a funnel-like shape to the distal tip region 356 (e.g., as shown in FIG. 11). This may help to guide the occlusive implant 26 into the distal tip region 356 (e.g., by actuating the core wire 24), for example when it is desirable to recapture and/or reposition the implant. The funnel-like shape may help to reduce damage to the delivery sheath 314 during a recapture and/or repositioning process. In some instances, when the grooved distal section 358 is in the expanded configuration, the grooves 366 and/or folds 364 may be smoothed. When shifting back to the first configuration, the grooved distal section 358 may be configured so that the folds 364 are re-formed or remembered. Shifting back and/or “remembering” the first configuration may be aided by the grooved distal section 358 including a shape memory material (e.g., a shape memory polymer and/or metal), by the resilience and/or elastic properties of the grooved distal section 358, combinations thereof, and/or the like.

In some instances, the distal tip regions 156, 256, 356 disclosed herein may include one or more axial slits or cutouts. The slits may form or otherwise define arm segments or flaps. The flaps may be configured to aid in expansion of the distal tip regions 156, 256, 356. In other words, the flaps may allow the distal tip regions 156, 256, 356 to open up or expand into an enlarged shape/configuration (e.g., which may resemble a funnel). Any of the distal tip regions 156, 256, 356 may include slits and flaps, as appropriate, even if not expressly illustrated.

The materials that can be used for the various components of the delivery system 10 (and/or other delivery systems disclosed herein) may include those commonly associated with medical devices. For simplicity purposes, the following discussion makes reference to the delivery system 10. However, this is not intended to limit the devices and methods described herein, as the discussion may be applied to other delivery systems disclosed herein.

The delivery system 10 and/or other components of the delivery system 10 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the delivery system 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the delivery system 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the delivery system 10 to achieve the same result.

In some embodiments, a degree of Magnetic Resonance Imaging (MRI) compatibility is imparted into the delivery system 10. For example, the delivery system 10, or portions thereof, may be made of a material that does not substantially distort the image and create substantial artifacts (e.g., gaps in the image). Certain ferromagnetic materials, for example, may not be suitable because they may create artifacts in an MRI image. The delivery system 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A delivery device for an occlusive implant, the delivery device comprising:
a delivery sheath having a proximal region and a distal tip region;
a reinforcing member extending along the proximal region;
wherein the distal tip region has a grooved distal section, the grooved distal section being configured to shift between a delivery configuration and an expanded configuration;
a core member slidably disposed within the delivery sheath; and
an occlusive implant releasably coupled to the core member.

2. The delivery device of claim 1, wherein the grooved distal section is fluted.

3. The delivery device of claim 1, wherein the grooved distal section includes one or more pleats.

4. The delivery device of claim 1, wherein the grooved distal section includes one or more folds.

5. The delivery device of claim 1, wherein the grooved distal section includes one or more folds when in the delivery configuration.

6. The delivery device of claim 1, wherein the grooved distal section includes one or more axial slits.

7. The delivery device of claim 1, wherein the proximal region includes a proximal outer diameter and wherein the grooved distal section has distal outer diameter that is substantially the same as or smaller than the proximal outer diameter when in the delivery configuration.

8. The delivery device of claim 1, wherein the proximal region includes a proximal outer diameter and wherein the grooved distal section has distal outer diameter that is larger than the proximal outer diameter when in the delivery configuration.

9. The delivery device of claim 1, wherein the proximal region includes a proximal outer diameter and wherein the grooved distal section has distal outer diameter that is larger than the proximal outer diameter when in the expanded configuration.

10. A delivery device for an occlusive implant, the delivery device comprising:
a delivery sheath having a proximal region and a distal tip region;
wherein the distal tip region includes a grooved distal section;
wherein the grooved distal section is configured to allow the distal tip region to shift between a delivery configuration and an expanded configuration; and
a proximal reinforcing member disposed along the proximal region.

11. The delivery device of claim 10, wherein the grooved distal section is fluted.

12. The delivery device of claim 10, wherein the grooved distal section includes one or more pleats.

13. The delivery device of claim 10, wherein the grooved distal section includes one or more folds.

14. The delivery device of claim 10, wherein the grooved distal section includes one or more folds when in the delivery configuration.

15. The delivery device of claim 10, wherein the grooved distal section includes one or more axial slits.

16. The delivery device of claim 10, wherein the proximal region includes a proximal outer diameter and wherein the grooved distal section has distal outer diameter that is substantially the same as or smaller than the proximal outer diameter when in the delivery configuration.

17. The delivery device of claim 10, wherein the proximal region includes a proximal outer diameter and wherein the grooved distal section has distal outer diameter that is larger than the proximal outer diameter when in the delivery configuration.

18. The delivery device of claim 10, wherein the proximal region includes a proximal outer diameter and wherein the grooved distal section has distal outer diameter that is larger than the proximal outer diameter when in the expanded configuration.

19. The delivery device of claim 10, further comprising a core member slidably disposed within the delivery sheath and an occlusive implant releasably coupled to the core member.

20. A method for implanting an occlusive implant into a left atrial appendage, the method comprising:
advancing a delivery device to a position adjacent to the left atrial appendage, the delivery device comprising:
a delivery sheath having a proximal region and a distal tip region,
a reinforcing member extending along the proximal region,
wherein the distal tip region includes a grooved distal section, the grooved distal section being configured to shift between a delivery configuration and an expanded configuration,
a core member slidably disposed within the delivery sheath, and
an occlusive implant releasably coupled to the core member; and
advancing the core member such that the occlusive implant advances out from the grooved distal section.
